(19) United States
(12) Patent Application Publication
(10) Pub. No.: US 2003/0096760 A1
(43) Pub. Date: May 22, 2003

(54) Method of antagonizing the human SRC SH2 domain

(75) Inventors: Dennis S. Yamashita, King of Prussia, PA (US); Daniel F. Veber, Ambler, PA (US); Dennis A. Holt, Stowe, MA (US)

Correspondence Address: GLAXOSMITHKLINE, Corporate Intellectual Property - UW2220, P.O. Box 1539, King of Prussia, PA, 19406-0939, US

(73) Assignee: SmithKlineBeecham Corporation

(21) Appl. No.: 10/119,235

(22) Filed: Apr. 8, 2002

Related U.S. Application Data
(63) Continuation of application No. 09/142,184, filed on Sep. 2, 1998, now abandoned.
Application No. 09/142,184 is a 371 of international application No. PCT/US98/04699, filed on Mar. 10, 1998.
(60) Provisional application No. 60/040,658, filed on Mar. 10, 1997.

(51) Int. Cl. A61K 38/06; A61K 31/4025; A61K 31/366

(52) U.S. Cl. 514/18; 514/456; 514/422; 514/320

(57) ABSTRACT

Invented is a method of treating a bone resorption disease in a subject which comprises administering to the subject a therapeutically effective amount of a compound which forms a covalent bond or link to cys 185 of the src SH2 domain.

### BACKGROUND OF THE INVENTION

Mammalian bone is constantly undergoing bone remodeling, which is a dynamic process of bone resorption and bone formation. These processes are mediated by specialized cell types: bone formation is the result of the deposition of mineralized bone by osteoblast cells, and bone resorption is the result of the dissolution of bone matrix by osteoclast cells. Many bone diseases are brought about by an imbalance of bone formation relative to bone resorption. For instance, diseases such as osteoporosis and Paget's disease are characterized by a net loss of bone matrix. Thus, agents which inhibit bone resorption are useful for the treatment of such diseases.

An activated osteoclast resorbs bone by attaching to the bone matrix, and secreting proteolytic enzymes, organic acids and protons into the sealed compartment formed between its cell membrane and the bone matrix. The acidic environment and proteolytic enzymes effect the dissolution of bone in the sealed compartment to crest pits, or lacuna, in the bone surface, which are apparent when the osteoclast detaches from the bone.

A number of polypeptide growth factors and hormones mediate their cellular effects through a signal transduction pathway. Transduction of signals from the cell surface receptors for these ligands to intracellular effectors frequently involves phosphorylation or dephosphorylation of specific protein substrates by regulatory protein tyrosine kinases (PTK) and phosphatases. Tyrosine phosphorylation may be the primary, or possibly even the sole, indicator of signal transduction in multicellular organisms. Receptor-bound and intracellular PTKs regulate cell proliferation, cell differentiation and signaling processes in immune system cells.

Aberrant protein tyrosine kinase activity has been implicated or is suspected in a number of pathologies such as diabetes, atherosclerosis, psoriases, septic shock, bone loss, anemia, many cancers and other proliferative diseases. Accordingly, tyrosine kinases and the signal transduction pathways which they are part of are potential targets for drug design. For a review, see Levitzki et al. in Science 267, 1782-1788 (1995).

Many of the proteins comprising signal transduction pathways are present at low levels and often have opposing activities. The properties of these signaling molecules allow the cell to control transduction by means of the subcellular location and juxtaposition of effectors as well as by balancing activation with repression such that a small change in one pathway can achieve a switching effect.

The formation of transducing complexes by juxtaposition of the signaling molecules through protein-protein interactions are mediated by specific docking domain sequence motifs. Src homology 2 (SH2) domains, which are conserved non-catalytic sequences of approximately 100 amino acids found in a variety of signaling molecules such as non-receptor PTKs and kinase target effector molecules and in oncogenic proteins, play a critical role. The SH2 domains are highly specific for short phosphotyrosine-containing peptide sequences found in autophosphorylated PTK receptors or intracellular tyrosine kinases.

Approximately 60 proteins having distinct catalytic or other functional domains yet sharing conserved SH2 domains, conserved sequences of approximately 100 amino acids, have been identified. It is not known precisely which physiological responses in the body are controlled by each of these SH2 domains. Further, the SH2 domain-ligand/compound interactions are highly specific such that minor modifications in the structure of the ligand/compound will significantly alter the selectivity with which the ligand/compound binds to the various SH2 domains.

The consequences of non selective antagonism of SH2 domains can be quite severe. For example, although the src SH2 domain, the lck SH2 domain and the fyn SH2 domain are structurally similar, possessing a high degree of conservation between the domains, antagonism of the src SH2 domain is indicated herein as effecting bone resorption while antagonism of the lck SH2 domain or the fyn SH2 domain induces immunosuppression. The induction of immunosuppression would be undesirable in long term therapy for bone resorption disease.

It would be desirable to provide methods and compounds which allow the treatment of a bone resorption disease by antagonizing the src SH2 domain.

Disclosed herein is an improved method of antagonizing the human src SH2 domain.

### SUMMARY OF THE INVENTION

The present invention provides a method of treating a bone resorption disease in a subject which comprises administering to the subject a therapeutically effective amount of a compound which forms a covalent bond or link to cys185 of the src SH2 domain.

The present invention also provides a method of treating osteoporosis in a subject which comprises administering to the subject a therapeutically effective amount of a compound which forms a covalent bond or link to cys185 of the src SH2 domain.

The present invention also provides a method of impairing the function of osteoclasts in a subject which comprises administering to the subject an osteoclast function-inhibiting amount of a compound which forms a covalent bond or link to cys185 of the src SH2 domain.

The present invention also provides compounds and pharmaceutical compositions of these compounds which are useful in antagonizing the human SRC SH2 domain.

### DETAILED DESCRIPTION OF THE INVENTION

As used herein, the term “a bone resorption disease” means any disorder characterized by abnormal bone loss due to osteoclastic activity, preferably osteoporosis.

As used herein, the term “treating” and derivatives thereof means prophylactic or therapeutic therapy.

As used herein, the term “compound” means a peptide or chemical compound.

As used herein, unless other wise defined, the term “peptidomimetic” is as defined in J. Med. Chem. 1993, 36, 3039-3049.

As used herein, unless other wise defined, the term “src SH2 domain antagonists” means a compound which is capable of forming a covalent bond or link to cys185 of the src SH2 domain.

As used herein the term “cys185 of the src SH2 domain” refers to the Cysteine at the 185 position of the src gene following conventional numbering as described in Nature 1997, 385, 595-602. All of the src gene numbering references used herein follow conventional numbering as described in Nature 1997, 385, 595-602.

The present invention provides a method of treating a bone resorption disease in a subject which comprises administering to the subject a therapeutically effective amount of a compound which forms a covalent bond or link to cys185 of the src SH2 domain.

The invention also provides a method of treating osteoporosis in a subject which comprises administering to the subject a therapeutically effective amount of a compound which forms a covalent bond or link to cys185 of the src SH2 domain.

The invention also provides a method of impairing the function of osteoclasts in a subject which comprises administering to the subject an osteoclast function-inhibiting amount of a compound which forms a covalent bond or link to cys185 of the src SH2 domain.

The nonreceptor tyrosine kinase src is essential for resorption of bone by osteoclasts. The src homology-2 (SH2) domain of src controls its association with other signaling molecules through binding to short peptide motifs containing phosphotyrosine. Inhibition of these interactions blocks src-mediated signal transduction by preventing recruitment of src into receptor-effector complexes. In the human src SH2 domain, cysteine 185 (cys185) is located in the phosphotyrosine binding pocket, close to histidine 201, arg155, arg175 and lys203. Compounds which form a covalent bond or link to cys185 block the phosphotyrosine binding pocket of human src SH2 thereby irreversibly inhibiting human src SH2.

In a preferred aspect of the invention, the compound which forms a covalent bond or link to cys185 will also form a hydrogen bond with arg175 and have a hydrophobic interaction with the sidechain portion of lys203.

The human src SH2 domain construct used in the present invention is described in Seq. ID No. 5. Seq. ID No. 5 uses a portion of src gene. As a reference, cys185 corresponds to cys67 in Seq. ID No. 5, his201 corresponds to his83 in Seq. ID No. 5, arg155 corresponds to arg37 in Seq. ID No. 5, arg175 corresponds to arg 57 in Seq. ID No. 5 and lys203 corresponds to lys85 in Seq. ID. No. 5.

Presently preferred compounds of this invention which form a covalent bond or link to cys185 of the src SH2 domain have the following Formula (I):

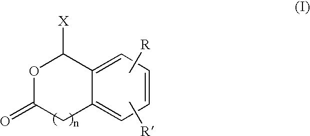

X=OR″, SR″, NR″R′″;

R″=H, methyl, alkyl;

R′″=CONH₂, CONHMe, CO NHalkyl, SONH₂, SONHMe, SONHalkyl, SO₂NH₂, SO₂NHMe, SO₂NHalkyl;

n=0,1,2;

R=H, CH₂CH(NHCOR″″)CONHR′″″, organic moiety;

R″″=glu-glu-ileu-glu-NH₂, peptide, peptidomimetic, alkyl, substituted alkyl, aryl, substituted aryl, heteroaryl, substituted heteroaryl;

R′=H, peptidomimetic; or

R,R′=fused ring system substituted with H or peptidomimetic. or a pharmaceutically acceptable salt, hydrate of solvate thereof.

Compounds of Formula I are included in the pharmaceutical compounds of the invention and used in the methods of the invention.

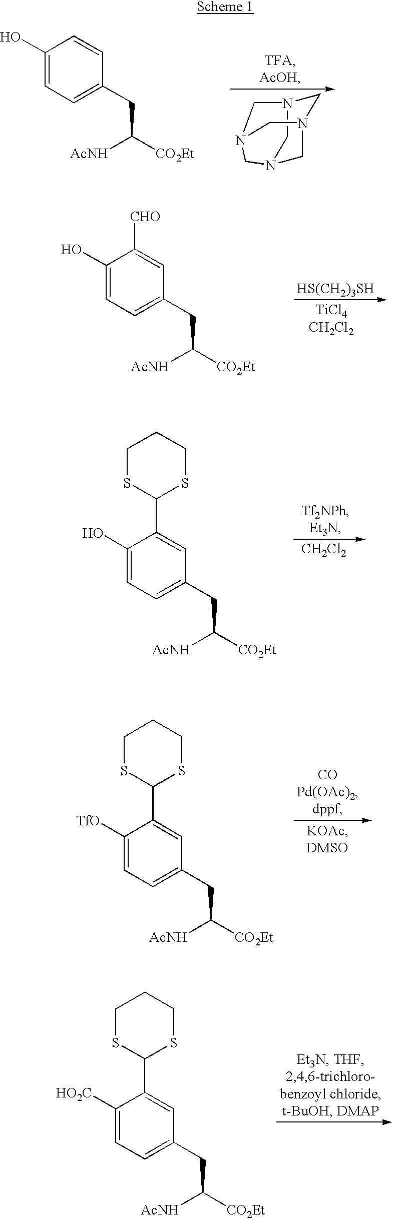

Scheme 1 depicts formation of (S)-alpha-(acetylamino)-1,3-dihydro-3-hydroxy-1-oxo-5-isobenzofuranpropanamido-glutamate-glutamate-isoleucine-glutamate-amine (Compound 1). N-acetyl tyrosine ethyl ester was formylated with hexamethylene tetramine (methenamine) in TFA, AcOH (J. Ind. Chem. 1987, 26B, 7071). Then the aldehyde was protected as its 1,3-dithiane (Tet. Lett. 1983, 24, 1289), then the phenol was triflated using N-phenyl trifluromethanesulfonimide. Palladium catalyzed hydroxy-carbonylation followed by esterfication using 2,4,6-trichloro benzoyl chloride gave the protected diester. Selective hydrolysis of the ethyl ester with sodium hydroxide in MeOH gave the desired amino acid analog, which was coupled to an immobilized peptide using standard coupling chemistry (HBTU (2-1H-benzotriazole-1-yl)-1,1,3,3-tetramethyluronium hexafluorophophate), N-methyl morpholine, DMF). Cleavage from the resin with concurrent deprotection of the t-butyl ester protective groups followed by a final deprotection of the aldehyde gave the target compound for testing in vitro.

Compounds of Formula I are prepared by methods analogous to the methods described in Scheme 1.

The inhibitory activity of compounds at the different human SH2 domains was determined in vitro using SH2 domains expressed as fusion proteins in E. coli as further described in detail in Example 2 below.

The data shown in accompanying Table 1 indicates that src SH2 domain antagonists will have significant efficacy in the fetal rat long bone (FRLB) assay. This in vitro activity is recognized in the art as correlating with efficacy in treating a bone resorption disease in vivo. This in vitro activity is also recognized in the art as correlating with efficacy in impairing the function of osteoclasts in vivo.

The present invention therefore provides a method of treating a bone resorption disease, which comprises administering a quantity of a src SH2 domain antagonists defined as herein in a quantity effective to inhibit bone resorption. The drug may be administered to a patient afflicted with a bone resorption disease or in danger of contracting a bone resorption disease by any conventional route of administration, including, but not limited to, intravenous, intramuscular, oral, subcutaneous, intradermal, and parenteral. The quantity effective to inhibit bone resorption is from about 0.001 mg per kg to about 10.0 mg per kg of subject body weight. The selected dose will be an efficacious, nontoxic quantity selected from about 0.001 mg per kg to about 10.0 mg per kg of subject body weight. The selected dose will be administered from about 1-6 times daily.

The method of treating a bone resorption disease disclosed in the present invention may also be carried out using a pharmaceutical composition comprising an src SH2 domain antagonists defined herein and a pharmaceutically acceptable carrier. The composition may contain between 0.05 mg and 500 mg of a src SH2 domain antagonist, and may be constituted into any form suitable for the mode of administration selected. Compositions suitable for oral administration include solid forms, such as pills, capsules, granules, tablets, and powders, and liquid forms, such as solutions, syrups, elixers, and suspensions. Forms useful for parenteral administration include sterile solutions, emulsions, and suspensions.

The present invention further provides a method of impairing the function of osteoclasts, which comprises administering a quantity of a src SH2 domain antagonists defined as herein in a quantity effective to inhibit bone resorption. The drug may be administered to a patient afflicted with a bone resorption disease or in danger of contracting a bone resorption disease by any conventional route of administration, including, but not limited to, intravenous, intramuscular, oral, subcutaneous, intradermal, and parenteral. The quantity effective to impair osteoclasts function is from about 0.001 mg per kg to about 10.0 mg per kg of subject body weight. The selected dose will be an efficacious, nontoxic quantity selected from about 0.001 mg per kg to about 10.0 mg per kg of subject body weight. The selected dose will be administered from about 1-6 times daily.

The method of impairing the function of osteoclasts disclosed in the present invention may also be carried out using a pharmaceutical composition comprising an src SH2 domain antagonists defined herein and a pharmaceutically acceptable carrier. The composition may contain between 0.05 mg and 500 mg of a src SH2 domain antagonist, and may be constituted into any form suitable for the mode of administration selected. Compositions suitable for oral administration include solid forms, such as pills, capsules, granules, tablets, and powders, and liquid forms, such as solutions, syrups, elixers, and suspensions. Forms useful for parenteral administration include sterile solutions, emulsions, and suspensions.

The drug may otherwise be prepared as a sterile solid composition which may be dissolved or suspended at the time of administration using sterile water, saline, or other appropriate sterile injectable medium. Carriers are intended to include necessary and inert binders, suspending agents, lubricants, flavorants, sweeteners, preservatives, dyes and coatings.

Optimal dosages to be administered may be readily determined by those skilled in the art, and will vary with the particular src SH2 domain antagonist in use, the strength of the preparation, the mode of administration, and the advancement of the disease condition. Additional factors depending on the particular patient being treated will result in a need to adjust dosages, including patient age, weight, diet, and time of administration.

The invention also provides for the use of a src SH2 domain antagonists in the manufacture of a medicament for use in the treatment of a bone resorption disease.

The invention also provides for the use of a src SH2 domain antagonists in the manufacture of a medicament for use in the treating osteoporosis.

The invention also provides for the use of a src SH2 domain antagonists in the manufacture of a medicament for use in inhibiting osteoclast function.

The invention also provides for a pharmaceutical composition for use in the treatment of a bone resorption disease which comprises a src SH2 domain antagonists.

The invention also provides for a pharmaceutical composition for use in the treatment of osteoporosis which comprises a src SH2 domain antagonists.

The invention also provides for a pharmaceutical composition for use in inhibiting osteoclast function which comprises a src SH2 domain antagonists.

No unacceptable toxicological effects are expected when the methods of the invention are utilized in accordance with the present invention.

Without further elaboration, it is believed that one skilled in the art can, using the preceding description, utilize the present invention to its fullest extent. The following Examples are, therefore, to be construed as merely illustrative and not a limitation of the scope of the present invention in any way.

### EXPERIMENTAL DETAILS

### EXAMPLE 1

### Preparation of (S)-alpha-(acetylamino)-1,3-dihydro-3-hydroxy-1-oxo-5-isobenzofuranpropanamido-glutamate-glutamate-isoleucine-glutamate-amine (Compound 1)

a) N-Acetyl-3-formyl-tyrosine ethyl ester

Hexamethylene tetraamine (Aldrich, 25 g, 178 mmol) was added to a solution of N-acetyl tyrosine ethyl ester mono hydrate (Aldrich, 10 g, 37.1 mmol) in TFA (30 ml) and AcOH (30 ml) and the reaction was heated to 80 degrees C. for 4.5 h. The reaction was cooled to RT, then H₂O (200 ml) was added and the reaction mixture was extracted with EtOAc (3×200 ml). The combined organics were dried with magnesium sulfate, filtered, concentrated in vacuo, and chromatographed (silica gel, 5% MeOH/CH₂Cl₂) to yield the title compound as a white solid (4.5 g, 46% yield): MS ES M+H⁺=280.

b) N-Acetyl-3-(1,3-dithiane)-tyrosine ethyl ester

A solution of TiCl₄ (Aldrich, 1.0 M, 1.4 ml, 1.4 mmol) was added dropwise to a solution of 1,3-propanedithiol (148 mg, 1.4 mmol) and N-Acetyl-3-formyl-tyrosine ethyl ester (400 mg, 1.4 mmol) in CH₂Cl₂ (7.0 ml) at 0 degrees C. The reaction was stirred for 2 h, then saturated aqueous NaHCO₃ (10 ml) was added, and the reaction mixture was extracted with EtOAc (3×20 ml). The combined organics were dried with magnesium sulfate, filtered, concentrated in vacuo, and chromatographed (silica gel, 5% MeOH/CH₂Cl₂) to yield the title compound as a white solid (420 mg, 81% yield): MS ES M+H⁺=370, M+Na⁺=392.

c) N-Acetyl-3-(1,3-dithiane)-4-triflyl-phenylalanine ethyl ester

N-phenyl trifluromethanesulfonimide (Aldrich, 1.0 g, 2.8 mmol) was added to a solution of N-acetyl-3-(1,3-dithiane)-tyrosine ethyl ester (1.0 g, 2.7 mmol) and triethyl amine (0.41 ml, 3.0 mmol) in CH₂Cl₂ (9.0 ml) at RT, and the reaction was stirred overnight. The reaction was diluted with H₂O (20 ml), then the reaction mixture was extracted with EtOAc (3×20 ml). The combined organics were dried with magnesium sulfate, filtered, concentrated in vacuo, and chromatographed (silica gel, 5% MeOH/CH₂Cl₂) to yield the title compound as a beige solid (1.14 g, 84% yield): MS ES M+H⁺=502, M+HCO₂^{-=546.}

d) N-Acetyl-3-(1,3-dithiane)-4-carboxy-phenylalanine ethyl ester

Palladium (II) acetate (11 mg, 0.215 mmol) was added to a mixture of N-acetyl-3-(1,3-dithiane)-4-triflyl-phenylalanine ethyl ester (540 mg, 1.07 mmol), 1,1′=bis-(diphenylphosphino)ferrocene (Aldrich, 119 mg, 0.215 mmol), potassium acetate (409 mg, 4.31 mmol) in DMSO (10.0 ml). The reaction mixture was heated to 80 degrees C., then carbon monoxide was bubbled through the solution for 10 minutes. Then, the reaction was stirred overnight under a balloon of carbon monoxide. The reaction was cooled to RT, diluted with H₂O (30 ml), extracted with EtOAc (4×30 ml). Then, the combined organics were dried with magnesium sulfate, filtered, concentrated in vacuo, and chromatographed (silica gel, 5% MeOH/CH₂Cl₂) to yield the title compound as a white solid (320 mg, 76% yield): MS ES M+H⁺=398, M−H⁻=396.

e) N-Acetyl-3-(1,3-dithiane)-4-(t-butyl-carboxylate)-phenylalanine ethyl ester

2,4,6-Trichlorobenzoyl chloride (Aldrich, 0.165 ml, 1.1 mmol) was added to a solution of N-acetyl-3-(1,3-dithiane)-4-carboxy-phenylalanine ethyl ester (420 mg, 1.1 mmol), triethyl amine, 0.295 ml, 2.1 mmol) in THF (5.3 ml) and the reaction was stirred for 0.25 h. Then, t-butanol (0.2 ml, 2.1 mmol) was added followed by 4-dimethyl amino pyridine (DMAP) (258 mg, 2.11 mmol) and the reaction was stirred overnight. The reaction mixture was loaded onto a chromatography column (silica gel, 5% MeOH/CH₂Cl₂) to yield the title compound as a white solid (250 mg, 55% yield): MS ES M+H⁺=454.

f) N-Acetyl-3-(1,3-dithiane)-4-(t-butyl-carboxylate)-phenylalanine

Aqueous sodium hydroxide (Aldrich, 1.0 N, 0.55 ml, 0.55 mmol) was added to a solution of N-acetyl-3-(1,3-dithiane)-4-(t-butyl-carboxylate)-phenylalanine ethyl ester (250 mg, 0.55 mmol) in MeOH (1.5 ml) and the reaction was stirred overnight. The reaction mixture was then diluted with AcOH (1.0 ml) and H₂O (10 ml) and the reaction mixture was extracted with EtOAc (4×30 ml). Then, the combined organics were dried with magnesium sulfate, filtered, concentrated in vacuo, and chromatographed (silica gel, 5% MeOH/CH₂Cl₂) to yield the title compound as a white solid (120 mg, 51% yield): MS ES M+H⁺=426, M+NH₄⁺=443, M−H⁻=424.

g) γ-t-butyl-glutamate-γ-t-butyl-glutamate-isoleucine-γ-t-butyl-glutamate-Rink Resin

The title peptide was prepared by standard solid-phase chemistry on a Symphony Multiple Peptide Synthesizer (Rainin) using standard FMOC protected amino acids (2×1.5 h, 6 equivalents using HBTU (2-1H-benzotriazole-1-yl)-1,1,3,3-tetramethyluronium hexafluorophophate)/N-methyl morpholine in DMF coupling conditions) and 20% piperidine/DMF deprotection conditions (10 min) starting with Rink Amide resin (Nova, 0.3 mmol/g, PS/1% DVB, 100-200 mesh, H. Rink Tet. Lett. 1987, 28, 3787).

h) N-Acetyl-3-(1,3-dithiane)-4-(t-butyl-carboxylate)-phenylalanine-γ-t-butyl-glutamate-γ-t-butyl-glutamate-isoleucine-γ-t-butyl-glutamate-Rink Resin

HBTU (2-1H-benzotriazole-1-yl)-1,1,3,3-tetramethyluronium hexafluorophophate) (61 mg, 0.16 mmol) was added to a slurry of N-Acetyl-3-(1,3-dithiane)-4-(t-butyl-carboxylate)-phenylalanine (68 mg, 0.16 mmol), γ-t-butyl-glutamate-γ-t-butyl-glutamate-isoleucine-γ-t-butyl-glutamate-Rink resin (200 mg, 0.08 mmol), N-methyl morpholine (0.023 ml, 0.24 mmol) in DMF (5.0 ml) and was shaken at RT for 48 h. The reaction mixture was filtered, washed with DMF (300 ml), then CH₂Cl₂ (300 ml), and was dried under vacuum overnight. The resin was tested Kaiser ninhydrin negative consistent with quantitative coupling to give the title compound.

i) N-Acetyl-3-(1,3-dithiane)-4-(t-butyl-carboxylate)-phenylalanine-glutamate-glutamate-isoleucine-glutamate-amine

N-Acetyl-3-(1,3-dithiane)-4-(t-butyl-carboxylate)-phenylatanine-γ-t-butyl-glutamate-γ-t-butyl-glutamate-isoleucine-γ-t-butyl-glutamate-Rink resin (100 mg, 0.04 mmol) was added to a solution of 95% TFA/H₂O and was shaken 4 h at RT. The reaction mixture was filtered, diluted with 100 ml cold ether and the precipitate was collected, dissolved in AcOH (5 ml), frozen to −78 degrees C., and lyophilized to give the title compound as a white, fluffy solid: MS ES M+H⁺=869, M−H⁻=867.

j) (S)-alpha-(acetylamino)-1,3-dihydro-3-hydroxy-1-oxo-5-isobenzofuranpropanamido-glutamate-glutamate-isoleucine-glutamate-amine

N-Acetyl-3-(1,3-dithiane)-4-(t-butyl-carboxylate)-phenylalanine-glutamate-glutamate-isoleucine-glutamate-amine (5 mg, 0.006 mmol) was dissolved in 90% acetone/H₂O (0.3 ml), then N-chlorosuccinimide (5 mg, 0.037 mmol) and silver perchlorate (10 mg, 0.048 mmol) were added and the reaction was stirred 10 minutes at RT. The reaction mixture was chromatographed (C₁₈ reverse phase silica, MeCN, H₂O), and the UV active fractions were combined, concentrated in vacuo, and dissolved in MeOH (0.2 ml). Cold ether was added and the precipitate was collected, washed with ether, dissolved in AcOH, frozen to −78 degrees C., and lyophilized to produce a white, fluffy solid (3 mg, 64% yield): MS ES M+H⁺=M+Na⁺=801, M−H⁻=777.

### EXAMPLE 2

### Protocol for the Determination of the Potency of src SH2 Domain Antagonists

The inhibitory activity of compounds at the human src SH2 domain was determined in vitro using the human src SH2 domain expressed as fusion proteins in E. coli.

The fusion protein containing the human SH2 domain was expressed as the general sequence: DET1-DET2-spacer-ek-SH2, where DET1, DET2, spacer, ek and SH2 are as described below. DET1 (“defined epitope tag 1”) (SEQ ID NO: 1) is an 11 amino acid sequence found in the Human Immunodeficiency Virus Type 1 (HIV-1) envelope protein gp120 (or gp160). Monoclonal antibodies to various epitopes of HIV-1 gp120 (or gp160) are known in the art, see, for example U.S. Pat. No. 5,166,050. One preferred example is monoclonal antibody 178.1 (see, e.g., Thiriart et al., J. Immunol., 143:1832-1836 (1989)), which was prepared by immunization of mice with a yeast-expressed HIV-1 gp160 molecule from strain BH10 (Ratner et al., Nature, 313:277-284 (1985)). This tag was used for detection of expression (by Western blot), for purification of the protein (by affinity chromatography), and for configuring assays in which the fusion protein was captured or immobilized using the 178.1 antibody. DET2 is a hexa-histidine sequence tag (SEQ ID NO: 2) which binds to nickel-containing resins and was used for purification purposes. Spacer (SEQ ID NO: 3) was utilized to design a BamH1 restriction site at the indicated position of the construct. The term -ek—refers to a recognition sequence (SEQ ID NO: 4) for the enterokinase protease which provides for the optional removal of the tags from the SH2 domain, thus producing an SH2 domain that contains no extraneous amino acids. SH2 domains which contain no extraneous amino acids are preferable to tagged protein for crystallography studies. SH2 refers to the human src SH2 domain or, as described below, a construct used in the preparation of the human src SH2 domain.

The DNA sequence encoding each DET1-DET2-spacer-ek-SH2 was designed such that the indicated restriction sites (BamH1 and XbaI) flank the spacer-ek-SH2 region, thereby allowing different spacer-ek-SH2 contructs to be readily substituted into any one of the vectors described in Procedures 2 or 3 below to create a DET1-DET2-spacer-ek-SH2 tagged protein. The DNA sequence encoding each DET1-DET2-spacer-ek-SH2 constructs was also designed such that the entire tagged SH2 domain can be moved as an Ndel-XbaI fragment into any expression vector containing an NdeI site at an appropriate distance downstream of E. coli transcription and translation regulatory sequences and a downstream cloning site compatible with XbaI. Although any suitable vector would yield similar results(e.g., pET-11a; Novagen, Inc.), the vector used in the instant experiments was E. coli expression vector pEA1KnRBS3. This vector is a derivative of the series of vectors described in Shatzman, A, Gross, M, and Rosenberg, M, 1990, “Expression using vectors with phage lambda regulatory sequences”, In: Current Protocols in Molecular Biology (F. A. Ausubel et al , eds.), pp. 16.3.1-16.3.11, Greene Publishing and Wiley-Interscience, N.Y. (hereinafter F. A. Ausubel et al.). The specific vector pEA1KnRBS3 is described in Bergsma et al, 1991, J. Biol. Chem. 266:23204-23214.

The procedures below describe the expression of chicken src and human src SH2 domains. First, the chicken src SH2 domain was expressed as DET1-DET2-spacer-SH2. Then, the other was inserted into this vector in place of chicken src to express protein in the form DET1-DET2-spacer-ek-spacer-SH2.

Procedure 1: Cloning and Expression of chicken src SH2 domain containing tags DET1 and DET2 (DET1-DET2-spacer-SH2).

A DNA sequence encoding the tagged protein DET1-DET2-spacer-SH2 was PCR amplified from a cDNA clone containing the chicken src gene (p5H; Levy et al 1986. Proc. Natl. Acad. Sci. USA 83:4228) by methods well known to those skilled in the art by using the following primers:

(SEQ ID NO: 17)

5′ TTCCATATGAAAAGTATTCGTATTCAGCGTGGCCCGGGCCGTCACCAC
CACCACCACCACGGGATCCCCGCTGAAGAGTGGTACTTT 3′

The underlined sites are an NdeI recognition site (5′) and a BamHI recognition site (3′).

5′GGAATTCTAGATTACTAGGACGTGGGGCAGACGTT 3′ (SEQ ID NO: 18)

The underlined region is an XbaI recognition site.

The PCR product was digested with NdeI and XbaI, followed by isolation of the digested fragment on an agarose gel. The fragment was ligated into NdeI-XbaI-digested pEA1KnRBS3 vector (Bergsma et al, supra) that had been agarose gel purified as a 6.5 kbp fragment. The ligation reaction was used to transform E. coli MM294cI⁺ (F. A. Ausubel et al., supra). A plasmid containing an insertion of the correct fragment was identified and confirmed by DNA sequencing. The resultant plasmid encodes DET1-DET2-spacer-SH2 under the control of the phage lamda P_{L}promoter and regulatory system. Plasmid DNA was purified from MM294cI⁺ and used to transform E. coli strain AR120. In this host strain, expression of the phage promoter can be induced by addition of nalidixic acid to the growing culture as described in F. A. Ausubel et al, supra. Nalidixic acid induction of AR120 containing this plasmid, followed by analysis of the cellular proteins on an SDS-polyacrylamide gel stained with Coomassie Blue (F. A. Ausubel et al., supra), resulted in appearance of a protein band with an apparent molecular weight of 15,000; this band was not seen in uninduced cells or in induced cells containing pEA1KnRBS3 lacking the PCR amplified fragment. Western blotting confirmed that the induced protein band reacted with the anti-DET1 monoclonal antibody 178.1.

Procedure 2: Cloning, expression and purification of human src SH2 domain containing tags and an enterokinase proteolytic cleavage site (DET1-DET2-spacer-ek-src SH2).

A DNA sequence encoding protein ek-src SH2 was PCR amplified from a cDNA clone containing the human src gene (c-src SH2 DNA sequence identical to that described in Takeya, T. and Hanafusa, H, 1983 Cell 32:881-890) using the following primers:

5′ CGGGATCCTGGACGACGACGACAAAGCTGAGGAGTGGTATTTT 3′ (SEQ ID NO: 19)

The underlined site is a BamHI recognition site.

5′ GGAATTCTAGACTATTAGGACGTGGGGCACACGGT 3′ (SEQ ID NO: 20)

The underlined region is an XbaI recognition site.

The PCR product was digested with BamHI and XbaI, followed by isolation of the digested fragment on an agarose gel. The fragment was ligated into BamHI-XbaI-digested expression vector containing the tagged chicken src gene DET1-DET2-spacer-SH2 described in Procedure 1 above. In that vector, the BamHI site is located between the coding regions for DET2 and SH2, and the XbaI site is located after the 3′ end of the SH2 coding region. The ligation reaction was used to transform E. coli MM294cI⁺. The construct DET1-DET2-spacer-ek-src SH2 was confirmed by DNA sequencing (SEQ ID NO: 5) and induced in E. coli strain AR120 as described in Procedure 1 above. A Coomassie-Blue-stained, Western-bolt-positive induced protein band with an apparent molecular weight of 16,000 was observed after nalidixic acid induction.

Cells were lysed at neutral pH by sonication in the presance of lysozyme. After centrifugation, the soluble extract was chromatographed on a Ni⁺⁺NTA column. After washing the column with equilibration buffer (Tris buffer pH 8 containing 0.5 M NaCl) and the same buffer containing 15 mM imidazole, the protein was eluted in highly purified form with 25 mM imidazole in equilibration buffer. The SH2 domain, purified in this fashion, was found to bind with high affinity in a specific, saturable fashion to the appropriate pY peptide in the “Binding Assays” described below, demonstrating that the tag did not interfere with function. This expressed fusion protein, DET1-DET2-spacer-ek-src SH2, was utilized in the “Binding Assays” described below in order to determine the specificity of compounds to selectively inhibit the human src SH2 domain.

Binding Assays: The potency of compounds at the human SH2 domain was determined based on the ability of such compound to selectively inhibit the SH2 domain from binding to its respective specific pY peptide.

The binding assay for the human SH2 domain and pY peptide was performed in an ELISA-based 96 well plate assay. In Millipore 96 well filter plates, hydrophilic Durapore® (pore size 0.65 um Cat. No. MADVN6550), was added 2 ul (50% suspension) of Protein-G Sepharose (available from Pharmacia of N.J. Cat. No. 17-0618-01) and 2 ul of 2 mg/ml of MAB178.1. 10 pmol of the subject SH2 domain fusion protein was added to one or more wells. The volume was brought to 100 ul with TBS-T (tris buffered saline plus 0.05% tween-20), incubated and shaken at room temperature for 1 hr. then washed 1× with TBS-T (4° C.). 90 ul of TBS-T was then added to each well. The specific pY biotinylated peptide was diluted to a concentration of 1.0 uM in TBS-T (this peptide can be obtained from Bachem Bioscience of Pennsylvania, Genosys Biotechnologies of Texas and California Peptide Research of California). 10 ul was aliquoted per well to yield a final concentration of 0.1 uM (approx. the K_{d} for the SH2 domain/peptide pair) and a final volume of 100 ul. The assay plates were incubated until equilibrium binding was attained (3 hr at 4° C. with shaking). The assay plates were washed 2× per well TBS-T (4° C.), then 100 ul of SABC (Strepavidin biotinylated horseradish peroxidase complex, available from the Zymed corporation of California cat. no. 93-0043), 1 drop reagent A (streptavidin) and 1 drop of reagent B (AH-biotin conjugated-horseradish peroxidase) per 10 ml of TBS-T, incubated at 37° C. for 30 minutes, then cooled to 4° C.) was added per well, then incubated at 4° C. for 30-60 minutes. The plates were then washed 4× with TBS-T (4° C.) (250 ul/well)/wash). 100 ul of 1 mg/ml OPD (o-phenyldiamine, Sigma Chemical Corporation, St. Louis Mo.) in Citrate Buffer was added per well. To stop development, 100 ul of 10% sulfuric acid was added per well. 150 ul from each well was then removed from the assay plate and placed in an ELISA plate. The A₄₉₀ of each ELISA plate was then determined.

### Determination of (IC₅₀) for Table I

Each control or compound was assayed in duplicate. The duplicates were averaged and the background subtracted and the maximal values with no inhibition were taken from the plate, then all other data points were expressed as a percent of the maximal value (or as % control). These % control data values were graphed in Kaleidagraph for Macintosh (Synergy Software). The curves on these graphs were nonlinear curve fitted with the following equation F(x)=Emax/(1+(k_{d}/conc)ˆ slope), wherein the k_{d} term represents the IC₅₀ for each of the curves.

### Determination of (Ki) for Table II

The Ki for respective compounds is calculated via the following equation (see reference). This expanded equation must be used under the conditions of this assay, due to the fact that the pY biotinylated peptide is not in vast excess concentration (100×) over the SH2 domain fusion protein. The IC₅₀ is an extrapolated value from a nonlinear curve fit using Kaleidagraph. Rtot and *D are known values for reagents input into the assay. KD generally must be experimentally determined for each combination of SH2 domain fusion protein and pY biotinylated peptide.

KI=(IC₅₀−Rtot+Rtot/2((*D/(KD+*D))+(KD/(KD+*D+Rtot/2)))/(1+*D/KD+Rtot/KD((KD+* D/2)/(KD+*D)))

KI=(uM)KD of competitor

IC₅₀=(uM) IC₅₀ for inhibitor, derived via nonlinear curve fit of competition selectivity assay data for the SH2 domain

Rtot=(uM) total SH2 domain concentration within 1 assay (microtitre plate) well

*D=(uM) concentration of specific pY and biotinylated peptide for the SH2 domain

KD=(uM)KD value for the specific pY and biotinylated peptide for the SH2 domain IC₅₀ is the concentration of inhibitor at which the response or signal is inhibited by 50%

KD is the dissociation constant for a ligand in a receptor/ligand interaction, normally equaling the concentration of ligand which is at ½ Vmax on a saturation binding curve>

The pY peptide ligand used in the above Binding Assays is as follows.

Biotinylated pY peptide ligand containing an aminocaproic acid (Aca) linker used for the human src SH2 domain.

Glu-Pro-Gln-pTyr-Glu-Glu-Ile-Pro-Ile-Tyr-Leu (SEQ ID NO: 13)

Results of Binding Assays:

Tables I and II illustrate the activity of SH2 antagonists at the human src SH2 domain.

TABLE I
ACTIVITY OF Src SH2 DOMAIN ANTAGONISTS AT
CLONED HUMAN Src SH2 DOMAIN (IC₅₀)

| Compound | Src |
|---|---|
| 1 | 0.25 uM |

NI—No inhibition observed out to 300 uM
X—not tested

TABLE II
ACTIVITY OF Src SH2 DOMAIN ANTAGONISTS AT
CLONED HUMAN Src SH2 DOMAIN (Ki)

| Compound | Src |
|---|---|
| 1 | XX |

NI—No inhibition observed below 1000 uM
X—not calculated
XX—not tested

### EXAMPLE 3

### Activity of src SH2 Domain Antagonists

The compounds of this invention which are antagonist of the human src SH2 domain are tested for their potency to inhibit osteoclast mediated bone resorption in the fetal rat long bone (FRLB) assay as described in Raisz L G (1965) J Clin Invest 44: 103-116, Stern PH et al., (1979) Skeletal Research: An experimental Approach. New York: Academic Press, 21-59 and Votta B J et al., (1994) Bone 15:533-538).

To perform the experiment timed-pregnant Sprague Dawley rats (Taconic Farms, Germantown, N.Y.) are injected subcutaneously with 200 μCi of ⁴⁵CaCl₂ on day 18 of gestation, housed overnight, then anesthetized with Innovar-Vet (Pittman-Moore, Mundelein. Ill.) and sacrificed by cervical dislocation. Fetuses are removed aseptically and radii and ulnae were dissected free of surrounding soft tissue and cartilaginous ends. The bones are cultured 18-24 hours in BGJ_{b} medium (Sigma) containing 1 mg/ml bovine serum albumin, then transferred to fresh medium and cultured for an additional 48 hours in the absence or presence of a compound which is an antagonist of the human src SH2 domain. ⁴⁵Calcium released into the medium and total calcium in the bones are measured by liquid scintillation spectrometry. Data is expressed as the % ⁴⁵calcium released from treated bones as compared to corresponding control bones. Statistical differences are assessed by employing a one-way analysis of variance for non-paired samples. Data are presented as mean±s.e.m., n=4. The experiment is generally repeated two times.

Data from the above experiment demonstrates the therapeutic effect of src SH2 domain antagonists in treating a bone resorption disease.

While the preferred embodiments of the invention are illustrated by the above, it is to be understood that the invention is not limited to the precise instructions herein disclosed and that the right to all modifications coming within the scope of the following claims is reserved.

What is claimed is:

1. A method of treating a bone resorption disease in a subject which comprises administering to the subject a therapeutically effective amount of a compound which forms a covalent bond or link to cys185 of the src SH2 domain.

2. A method of treating osteoporosis in a subject which comprises administering to the subject a therapeutically effective amount of a compound which forms a covalent bond or link to cys185 of the src SH2 domain.

3. A method of impairing the function of osteoclasts in a subject which comprises administering to the subject an osteoclast function-inhibiting amount of a compound which forms a covalent bond or link to cys185 of the src SH2 domain.

4. The method of claim 1 in which the compound as the following formula I: 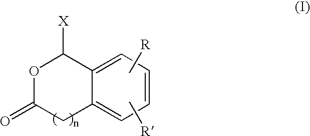
X=OR″, SR″, NR″R′″;
R″=H, methyl, alkyl;
R′″=CONH₂, CONHMe, CO NHalkyl, SONH₂, SONHMe, SONHalkyl, SO₂NH₂, SO₂NHMe, SO₂NHalkyl;
n=0,1,2;
R=H, CH₂CH(NHCOR″″)CONHR′″″, organic moiety;
R″″=glu-glu-ileu-glu-NH₂, peptide, peptidomimetic, alkyl, substituted alkyl, aryl, substituted aryl, heteroaryl, substituted heteroaryl;
R′=H, peptidomimetic; or
R,R′=fused ring system substituted with H or peptidomimetic. or a pharmaceutically acceptable salt, hydrate of solvate thereof.

5. The method of claim 2 in which the compound as the following formula I: 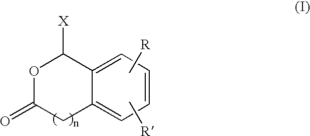
X=OR″, SR″, NR″R′″;
R″=H, methyl, alkyl;
R′″=CONH₂, CONHMe, CO NHalkyl, SONH₂, SONHMe, SONHalkyl, SO₂NH₂, SO₂NHMe, SO₂NHalkyl;
n=0,1,2;
R=H, CH₂CH(NHCOR″″)CONHR′″″, organic moiety;
R″″=glu-glu-ileu-glu-NH₂, peptide, peptidomimetic, alkyl, substituted alkyl, aryl, substituted aryl, heteroaryl, substituted heteroaryl;
R′=H, peptidomimetic; or
R,R′=fused ring system substituted with H or peptidomimetic. or a pharmaceutically acceptable salt, hydrate of solvate thereof.

6. The method of claim 3 in which the compound as the following formula I: 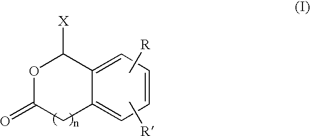
X=OR″, SR″, NR″R′″;
R″=H, methyl, alkyl;
R′″=CONH₂, CONHMe, CO NHalkyl, SONH₂, SONHMe, SONHalkyl, SO₂NH₂, SO₂NHMe, SO₂NHalkyl;
n=0,1,2;
R=H, CH₂CH(NHCOR″″)CONHR′″″, organic moiety;
R″″=glu-glu-ileu-glu-NH₂, peptide, peptidomimetic, alkyl, substituted alkyl, aryl, substituted aryl, heteroaryl, substituted heteroaryl;
R′=H, peptidomimetic; or
R,R′=fused ring system substituted with H or peptidomimetic. or a pharmaceutically acceptable salt, hydrate of solvate thereof.

7. The method of claim 1 in which the compound further forms a hydrogen bond with arg175 and have a hydrophobic interaction with lys203.

8. A pharmaceutical composition comprising a suitable pharmaceutical carrier and a compound as defined in claim 1.


SEQUENCE LISTING (10 sequences)

SEQ ID NO: 1 - protein, 11 aa, homo sapiens
KSIRIQRGPGR

SEQ ID NO: 2 - protein, 6 aa, homo sapiens
HHHHHH

SEQ ID NO: 3 - protein, 3 aa, homo sapiens
GIL

SEQ ID NO: 4 - protein, 5 aa, homo sapien
DDDDK

SEQ ID NO: 5 - protein, 130 aa, homo sapiens
MKSIRIQRGPGRHHHHHHGILDDDDKAEEWYFGKITRRESERLLLNAENPRGTFLVRESETTKGAYCLSVSDFDNAKGLNVKHYKIRKLDSGGFYITSRTQFNSLQQLVAYYSKHADGLCHRLTTVCPTS

SEQ ID NO: 6 - protein, 11 aa, homo sapiens
EPQYEEIPIYL

SEQ ID NO: 7 - protein, 87 aa, homo sapiens
TTCCATATGAAAAGTATTCGTATTCAGCGTGGCCCGGGCCGTCACCACCACCACCACCACGGGATCCCCGCTGAAGAGTGGTACTTT

SEQ ID NO: 8 - protein, 35 aa, homo sapiens
GGAATTCTAGATTACTAGGACGTGGGGCAGACGTT

SEQ ID NO: 9 - protein, 43 aa, homo sapiens
CGGGATCCTGGACGACGACGACAAAGCTGAGGAGTGGTATTTT

SEQ ID NO: 10 - protein, 35 aa, homo sapiens
GGAATTCTAGACTATTAGGACGTGGGGCACACGGT